(19) United States
(12) Patent Application Publication
(10) Pub. No.: US 2024/0197378 A1
(43) Pub. Date: Jun. 20, 2024

(54) SYSTEMS AND METHODS FOR THE MEASUREMENT OF THE STATE OF CURE OF PMMA IN A SURGICAL DISPENSING APPLICATOR

(71) Applicant: ORTHOPATENT, LLC, Cincinnati, OH (US)

(72) Inventors: Daniel A. FUNK, Cincinnati, OH (US); Quang-Viet NGUYEN, Aldie, VA (US); Avonley C. NGUYEN, Aldie, VA (US)

(21) Appl. No.: 18/538,527

(22) Filed: Dec. 13, 2023

Related U.S. Application Data
(60) Provisional application No. 63/432,871, filed on Dec. 15, 2022.

(51) Int. Cl. A61B 17/88

(52) U.S. Cl. CPC A61B 17/8833; A61B 17/8836

(57) ABSTRACT

The invention comprises a system for aiding in the in-situ determination of the state of cure (or progress of cure) in bone cement which is a composition that cures in an exothermic reaction and having a device which comprises a frequency sensor joined to a circuit and to an indicator that emits a signal in response to a current emitted to the circuit by the sensor to monitor a state of cure. The invention also relates to surgical methods using the system.

### FIELD OF THE INVENTION

The field of this invention is in the area of medical devices, and in particular, a convenient medical device systems for use in applying surgical cement as well as assessing the degree of cure of bone cement during surgery, as well as to methods of use of the devices, including surgical methods using the system.

### BACKGROUND OF THE INVENTION

The present invention addresses issues relating to devices used in the application of surgical cement as well as a method for the determination of the state of cure (or progress of cure) of bone cement or grout applied using the device in present surgical procedures. The invention or portions of the invention may also have an application to determination of viscosity states of other materials undergoing states of change, such as other adhesives or acrylates, bread dough or Portland cement, for example.

The state of the art for determining the state of cure of the PMMA (polymethylmethacrylate) intraoperatively is problematic, as it is generally determined either by direct palpation of the PMMA edge or by allowing extra PMMA not used in the surgical implantation to harden. This invention provides a quantitative solution to the issue of determination of cure during preparation for device implantation and following implantation, and further provides a method of surgery having an improved workflow enabled by a novel application system. The application system includes an applicator which contains bone cement for use during surgery and for which the desired state of cure for use can be determined by an integrated sensor.

Acrylic bone cement has been used in orthopedic surgery for over fifty years and is the standard of care for fixation of total joint arthroplasty. After mixing the liquid MMA (methyl methacylate) monomer with the powdered pre-polymer PMMA, the cement converts from liquid to solid by an exothermic reaction. Notably, the MMA monomer fumes are both foul smelling and potentially dangerous, and the exothermic reaction produces a substantial amount of heat, both issues presenting possible complications during the surgery. Moreover, the duration of full polymerization (i.e. “cure”) is variable and depends on multiple factors, including ambient temperature and ambient humidity. The ASTM Standard specification for full orthopedic cement curing in a testing environment is based on the temperature of the cement and the cure temperature. However, the changes in temperature are not reliable indicators of the progress of cure during the initial time period when the cement monomer and polymer are mixed and awaiting application as the temperature changes very little (circa 1° C.) during this period while the viscosity and cement ductility markedly changes. What is desired is a means of monitoring the cure state or progress of cure while the cement mixture awaits application and to know how much time is left before the cement mixture cannot be used anymore. If the cement has progressed too far, it will be too viscous to form so-called micro-interlocking interfaces with the bone to form a strong and durable bond.

One technique for improving this penetration is use of the mixed cement at the proper stage of cure which is presently difficult to determine and estimated based on surgical staff experience.

The question of suitable cure stage is influenced by a number of factors, including, for example, the ambient temperature, the exact composition of the cement, the ambient humidity, and mixing conditions. Additional factors that affect the clinical performance of the cement include the use of antibiotic additives, mixing methods, sterilization of the implants and instruments, temperature during handling, contamination by biological debris, mechanical properties, and state of the bone which receives it.

The “cure” also known as polymerization or cross-linking is initiated by an initiator and/or catalyst and generates heat in the form as an exothermic reaction. As previously mentioned, this heat is one sign of the cure process which is presently imprecisely observed to determine the state of cure subjectively. For example, the observation of a change in the surface gloss is indicative that the composition has reached the glass transition point which marks a significant point during the cure reaction.

Surgical cements generally are provided in two varieties for use in different procedural applications: medium viscosity (MV) and high viscosity (HV). Following mixing, all bone cements reach a higher viscosity state (“dough state” or “semi-cured state”) and MV cements reach that higher dough state somewhat later than HV cements and the techniques for use vary accordingly. Medium viscosity cement is sometimes classified as having dual phases i.e. low viscosity (prior to reaching its dough state) and medium viscosity (once the dough state has been reached). In contrast, high viscosity cements reach dough state quickly after mixing, and therefore do not have a low viscosity state. HV cement needs to be used immediately from the start of its dough time, to optimize its working time. The optimum time for cement application to the metal implant in joint replacement surgery, is just before the cement has reached this dough state (i.e. while the cement is tacky) to aid adherence to the implant. For high viscosity cements, the cement should be used as soon after mixing as practical. Knowing when these cements have reached their “point of no return” or “must use” state is important and heretofore, has not been something that can be reliably and quantitatively ascertained while the cement awaits dispensing in its application syringe.

Following are some useful definitions to understand the relevant terms and concepts involved in cement use and cure:

Glass Transition Temperature: glass transition temperature of a polymer is the temperature at which an amorphous polymer moves from a hard or glassy state to a softer, often rubbery or viscous state. This is marked by a change in the surface appearance from a shiny reflective surface to a dull or matter surface and is presently significant in the determination of when a cement is ready for use. However, it is also dependent on lighting conditions and line of sight, which can be an issue in a crowded operation room (OR).

Dough time: the change in physical condition of the cement which results from the initial polymerization of the liquid monomer with the powdered pre-polymer and identified by an increase in cement viscosity (in which it become stiffer and less runny), a change to a dull appearance, and reduced tackiness against surgical gloves.

Working time: sometimes referred to as “dough state” is the appropriate period of time for implanting the cement in the semi-cured “dough state” and prior to full cure or polymerization for a given ambient temperature according to its specific instructions for use provided in the use instructions.

Cement setting or curing time: is the time when the cement hardens as a result of fuller polymerization.

Working time/temperature chart: a chart contained in the use instructions that defines the available working time for a given ambient temperature. This is the optimal time period for application of the cement in its semi-cured state.

Ambient temperature: the temperature of the surrounding environment in the operating room. It can also refer to the temperature of the cement components prior to the release of heat from the exothermic reaction.

Final pressurization: the final application of pressure to securely seat the implant within the bone cement by means of bringing the joint, with the implant, into extension and holding it there until the cement cures.

Joint Preparation Instruments—Clearance: these instruments provide clearance around the implant for a cement mantle. Thus, removing a larger diameter of bone than the implant. Alternatively the joint may be prepared for Non-Clearance, commonly known as line to line instruments, which removes the same diameter of bone as the implant.

Presently, the use of bone cement, and in particular the assessment of the state of cure of bone cement during implant surgery requires a great deal of experience on the part of the surgical team, and is generally determined as discussed by blindly allowing the passage of time after mixing or by visual examination of a spare sample of mixed cement which is watched and sometimes touched for signs of cure, and specifically for a matte appearance to the surface, and after use by palpating the cement edge or by allowing the remainder cement to harden in vitro. These methods are imprecise, subjective, and unscientific and require time and experience to master any knowledge of cure. Moreover, intraoperative observation by surgeons has noted that in vivo cement appears to cure faster than the remaining excess cement in vitro. This is likely because the in vivo cement is in a warmer and more humid environment. Therefore, judging the state of cure after implantation through observation of an excess sample of cement can be mis-leading.

The state of cure during use directly influences the viscosity of the cement and the ease of application and fill as well as penetration into the environmental bone. It is critical that the cement have an appropriate amount of cure during its application for the proper structure to result after full cure. If it is too early, it is runny and hard to control, may lead to poor pressurization and reduced penetration, and will subject the bone to excess heat during further cure. This also carries the risk of higher heat discharge, which can harm the tissue, from the exothermic reaction of the polymerization. If it is too late, it is too stiff, and will may reduce the flow of the cement into the bone to inhibit penetration into the bone and potentially not conform or adhere within the environment and to the implant and also reduce the interfacial strength of the cement with the implant. Accurate determination of curing of the cement while it awaits application is important, as well as determining if it has fully cured after it has been applied to the bone and implanted device or structure.

The prior art has presented various solutions to the issues of monitoring or determining cure states during polymerization, some of which involves the use of sound waves to monitor the attenuation or impedance of the sound waves through a sample of the cement.

U.S. Pat. No. 8,347,723 to Questo et al. provides a technology using a sonic resonator having a transducer probe that physically contacts the sample to be tested to test the adhesive bond strength in a composite material. Ultrasonic waves are generated and sent to a probe tip and acoustical impedances between the probe and the material shows up as reflections signals. The present technology differs in the use two transducers in series in which an electromagnetic actuator generates a pressure excitation waved through the piezo crystal which is only used as a receiver. In this invention the sound waves travel into the cement in some stage of cure and the acoustic interface is analyzed to pick up a totally different generation of new frequencies of waves as the polymerization proceeds. The use of the generation of a new frequency wave is a key distinction and advantage over the Questo et al. prior art as new frequencies are readily detectable in noisy environments, such an operating room.

U.S. Pat. No. 9,297,789 to Djordjevic et al. also uses ultrasonic direct contact but uses a small reference notch in the probe to measure the background signal so that a ratio can be taken to reduce noise. This technology does not use a second transducer in series, instead it also looks at interface impedances. Again, this technology does not suggest using the generation of new frequencies in the cement to monitor the state of cure. U.S. Pat. No. 5,145,250 to Planck et al relates to sensing the temperature and pressure of a polymer during cure inside an automated cement mixer. US Patent Publication No. 2009/0112365 to Orr et al. uses a defined property of the cement, and including one or more of the ambient temperature, the ambient humidity, the viscosity of the cement or the speed of sound through the cement which is measured using ultrasonic transducers to monitor the cure state of the cement. US Patent Publication No. 2021/0302374 to Jack looks for potential fail points in composite laminates and uses sound to create an image of the internal morphology of an adhesive layer or interface. This reference is similar to taking a sonogram of a test sample to see changes in hardness via an acoustic reflection from changes in acoustic wave impedance. A reference entitled “Ultrasonic characterization of the mechanical properties and polymerization reaction of acrylic-based bone cements” by Dunne et al, DOI: 10.1243/09544119JEIM168 2006 teaches using the application of sound waves to a sample immersed in water (similar to sonar) to detect the change in the acoustic impedance via reflection. It requires a liquid interface to give time of flight information of the cement sample.

None of the prior art uses the detection of the shift of resonant frequencies during cure, nor do they teach electromagnetic voice coil transducers (Tx) in series with piezo (Rx) to detect the change in cure via monitoring of the resonant frequency. The present invention requires little signal analysis or processing since detection of a resonant frequency shift is arbitrarily very sensitive and immune to noise and background interferences, and since the amplitude of the frequency or a comparison of the relative amplitude of frequencies can be used to determine an amount of cure of the monitored sample.

It has been shown that is desirable to be able to provide an improved application device which embodies a method to monitor both the degree and the rate of cure of the PMMA cement or grout so as to enable the optimal state for use and further to determine the progress of the cure and when the full cure has occurred to allow proper final pressurization and to document that hardening of the PMMA has been achieved during surgery. More accurate determination provides for decreased surgical time and better protection from PMMA implant bond breakage.

The present invention thus relates to such an application device which contains a quantity of material, notably bone cement, and which includes a sensor that can monitor the state of cure of the material and determine when it has reached a desired state. For specific use of bone cement, this state is one that is suitable for application during surgery. In addition, the sensor can be used to monitor when the material is no longer suitable for use.

The present technique to assess for cement cure at the time of surgery is imprecise. The devices and methods of the present invention are unique and novel. When used in combination these methods and devices would help facilitate surgical efficiency, provide assurance to the operating team as to the state of cement cure during the procedure and protect the knee from early motion until full cement cure. Presently, determining the state of cure of the cement intraoperatively is, at best, imprecise. The state of the art of using remainder in vitro cement to determine cure has changed little from the early days of arthroplasty. Waiting for the extra cement to cure is a time-honored artistic ritual for surgeons and staff.

### SUMMARY OF THE INVENTION

The present invention addresses issues relating to cure of bone cement or grout which is used in conjunction with present surgical procedures. In particular, the present invention provides an application system containing a quantity of cement for use and having a sensor which utilizes sound waves to allow the user to monitor the state of a reaction of the cement in an applicator and to receive an alert when a set condition has occurred.

Optionally, the system can be a closed system in which the components are provided individually, fully mixed to form a sufficiently homogenous mixture, and allowed to cure while being monitored to determine a desired state of cure for use, and which can subsequently allow for application, within the same system. As a further embodiment, the system could include a sterile and disposable dispenser having a two or more component feed with a spiral plunger that drives the components in a mixing chamber to an application nozzle where the mixed material in the mixing chamber is in communication with the sensor of the present invention to determine if the material is suitable for use.

The system enables the user to place a wave sensor in direct or indirect contact with the cement, such as within the mixing and/or application vessel and after implantation during major orthopedic surgery, such as, for example total knee replacement surgery. This system comprises a tester that includes an acoustic transducer which acts as a signal transmitter which is in series with an acoustic and/or ultrasound sensor which acts as a signal receiver to monitor a change in the frequency or frequencies in response of the signal as an indication of cure of the cement. The resultant sensor is joined to a controller that receives the data generated by the sensor in use, monitors it for the noted change or changes, and accordingly, alerts the user, such as by a visual or audible sign that the desired state has been reached.

A method of using the system is accomplished such as by the use of a fixture and/or drill guide which is designed to work within an established work flow method, and by using a tester (sensor probe) including a mechanical vibrational frequency sensor such as an electro-mechanical transducer that imparts mechanical vibrations into a solid probe that makes contact with the cement to be monitored. The mechanical vibrations of the transducer have a characteristic resonant acoustic frequency spectrum that is modified by the state of the cement that it touches. This spectrum is obtained via a Fast Fourier Transform (FFT) and the changes in the cement cure state hardness for example, at the interface of the cement and probe tip or through the cement or at the interface of the cement and the container in which it is contained, produces a distinctly new frequency peak that can be unambiguously detected, or a shift in an existing resonant frequency. The FFT spectra of the sensor assembly can determine the moment that the cement has reached a defined condition, such as a desired cured state by generation of a new frequency peak. The detection of the generation of new frequency peaks is typically much more sensitive than detection of a linear change in some physical property because frequency generation can be arbitrarily filtered and amplified greatly for a high signal to noise ratio. The signal that results can be processed using either and analog or digital means. Typically, hardness changes registered as resonant frequency peak shifts which can be analyzed using computer-based algorithms in either hardware such as digital signal processing (DSP), field programmable gate arrays (FPGA), or in software (C/C++ etc).

The concept of a cement hardness sensor that monitors the state of the chemical reaction progress for PMMA cement in an applicator and which is awaiting to be applied is novel. The analysis of the generated acoustic resonant frequency spectra from the in-vivo measurement to determine unambiguously, the state of the cement cure point is also novel in that it uses a unique conditional related to the cement cure state hardness, but does not depend on the absolute temperature, which can vary greatly depending on the application and environment in which the cement is used and which is also free from the issues relating to extraneous environmental noise.

A method of using the system is accomplished within an established workflow method or advantageously in a novel workflow which eliminates or reduces the transfer of the cement and/or of the cement components or by-products, and by using the tester including a wave sensor that converts a wave response into a signal that can be processed using either analog or digital means. Typically, these changes are registered as voltage changes which can be analyzed using analog comparator circuits to provide outputs to a logic circuit operatively joined to an indicator, such as a display, light, alarm, haptic indicator or robotic surgical device. Alternatively, the wave signals can be digitized using an analog to digital converter (ADC) and the digital representation of the wave or wave change is analyzed to drive a logical indicator. The concept of a surgical method using a sound sensor that monitors the frequency response to monitor the state of the chemical reaction progress of a material in a closed system that can be used for application of the material is novel.

### BRIEF DESCRIPTION OF THE DRAWINGS

The invention will be better understood and other features and advantages will become apparent by reading the detailed description of the invention, taken together with the drawings, wherein:

FIG. 1 is an illustration of the tester in use with an application syringe for the determination of the cement cure progress and showing a circuit diagram with the electronic components in accordance with the present invention;

FIG. 2 shows a photograph of a cement cure progress sensor test apparatus used to determine and characterize the variation of the resonance frequency shift as a function of time and temperature;

FIG. 3 is series of FFT spectra of the resonant frequency shift as a function of cure time and temperature (indicated in the legend);

FIG. 4 is a graph showing the measured frequency shift and temperature as a function of time for sample number 11 in a series of tests using the apparatus depicted in FIG. 2;

FIG. 5 is another graph showing the measured frequency shift and temperature as a function of time for sample number 12 in a series of tests using the apparatus depicted in FIG. 2;

FIG. 6 is another graph showing the measured frequency shift and temperature as a function of time for sample number 13 in a series of tests using the apparatus depicted in FIG. 2;

FIG. 7 shows a graph of all the data collected from all tests to illustrate the concept of an average frequency shift rate (Hz/sec) for 19 sets of data and how they have a consistent slope when only the first 400 seconds are considered. During this time, the temperature of the sample changes only about 1 deg C.; and

FIG. 8 shows a syringe style applicator in which the components of the cement are provided individually, subsequently exposed to each other and mixed, and finally monitored as a mixture to determine an appropriate state of cure.

### DETAILED DESCRIPTION OF THE INVENTION

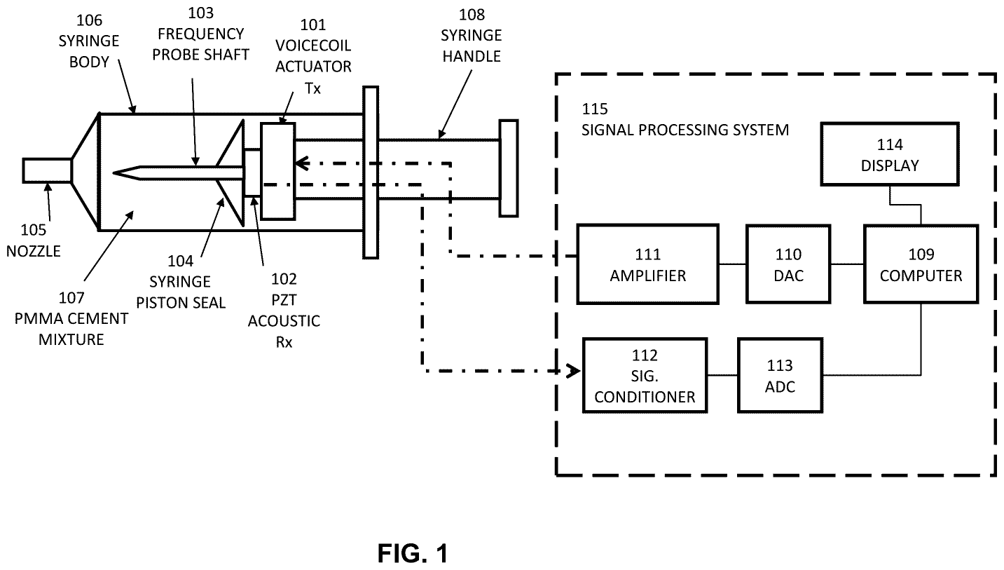

The present invention operates on the principle of the measurement of the resonance frequency of a mechanical structure in contact with the cement sample undergoing cure. As the cement cures, its viscosity changes and this affects the resonance frequency of the mechanical structure in contact with the cement. This mechanical structure is typically a probe tip that is in mechanical communication with a transducer that actuates vibrations on the probe and a transducer that senses those same vibrations. By monitoring the resonant frequency of the vibrations of the probe tip, one can ascertain in a quantitative way, the progress of the cure of the PMMA cement. FIG. 1 shows a voice coil actuator 101 that transmits mechanical oscillations through a piezoelectric transducer 102, through a probe shaft 103 that penetrates a syringe piston seal 104. The voice coil transducer 101 is mounted on the other side to the syringe handle 108 which resides inside the bore of a syringe body 196. The PMMA cement mixture that is being monitored is contained inside the syringe body 106 and the syringe piston seal 104, while in mechanical contact with the frequency probe shaft 103. Electrical wires connect the voice coil actuator 101 and piezoelectric transducer 102 to a signal processing system 115. The signal processing system 115 comprises an amplifier 111, which provides the excitation for the voice coil actuator 101, and the amplifier 111 is driven by a digital to analog converter (DAC) 110, that is controlled by a computer 109, which is also connected to an analog to digital converter (ADC) which receives the signal from the piezoelectric transducer 102. The ADC signal is captured and analyzed by software in the computer 109 where FFT's are performed on the data streams to generate a binary representation of the frequency spectrum so that the peak frequency position can be extracted.

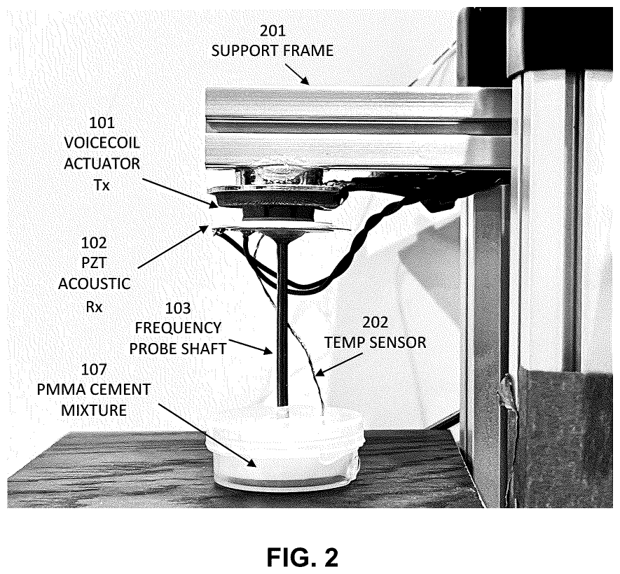

FIG. 2 shows a photograph of the test apparatus with a voice coil actuator 101, a piezoelectric transducer 102, and a probe shaft 103 in contact with a PMMA cement sample 107 held in a small plastic sample holder fitted with drilled holes to allow for the probe tip 103 to be in contact with the cement, and for a temperature sensor 202 to be in contact with the cement. These items are mounted to a support frame 101 to position the sensor with respect to the PMMA sample. The tests all utilized 2.0 grams of monomer and 2.0 grams of polymer that has been mixed in a separate container and then transferred to the sample holder.

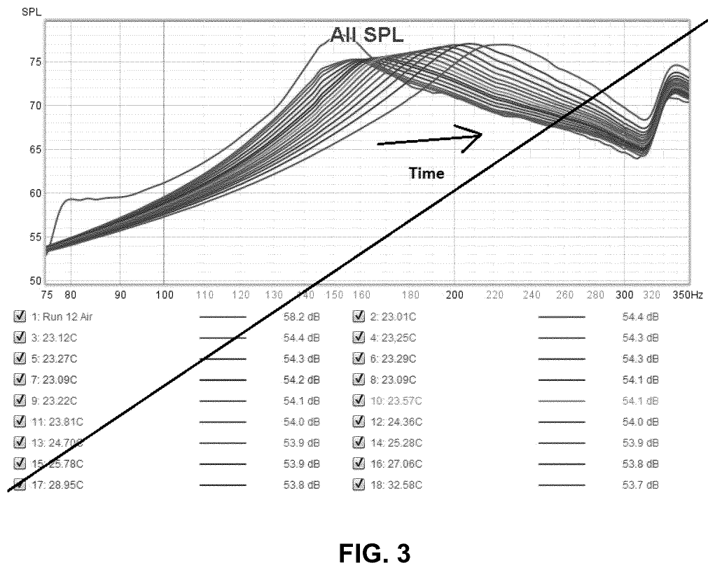

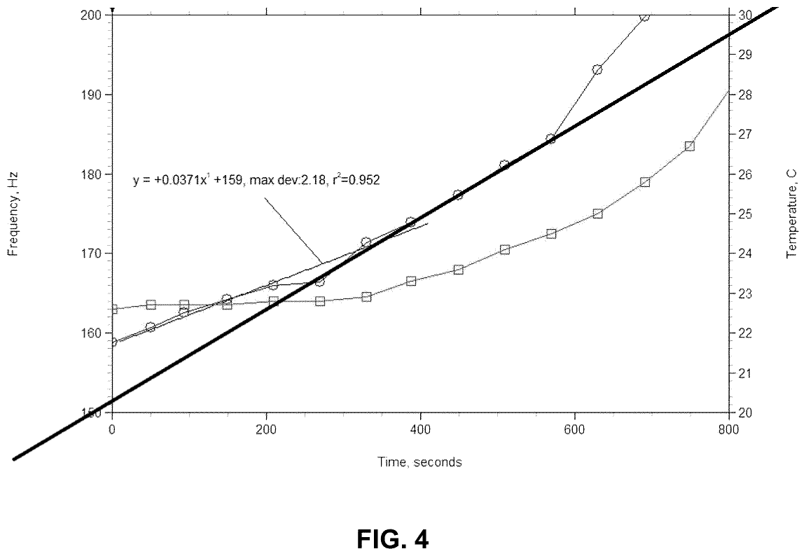

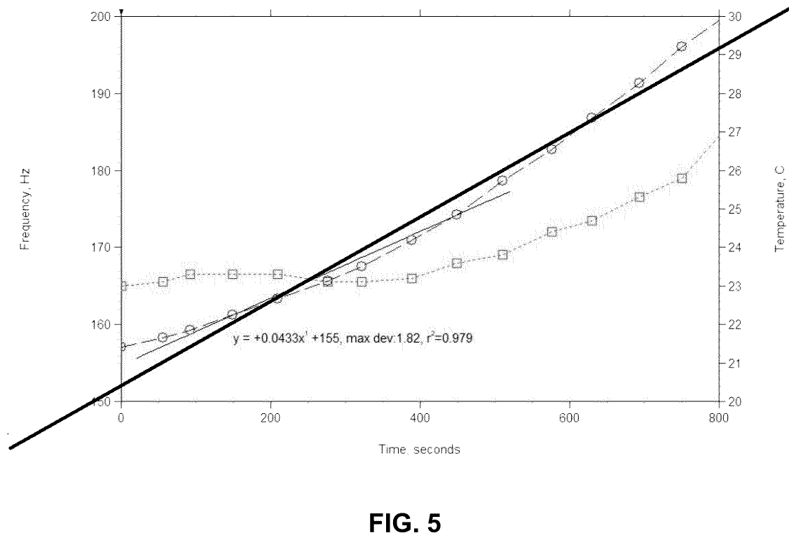

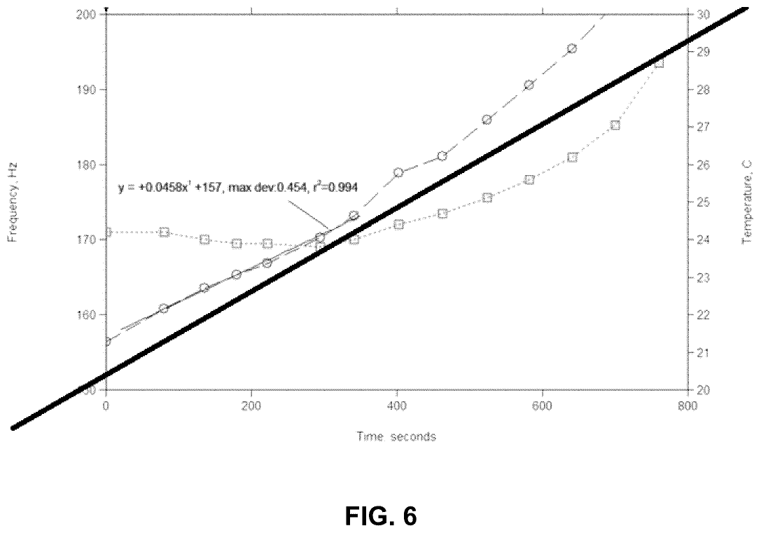

FIG. 3 shows a series of frequency spectra collected by excitation of the voice coil transducer and measuring the signal generated by the piezoelectric transducer. This signal is digitized using an ADC and the digital representation of the time series signal is converted to the frequency domain using an FFT in software. The FFT spectra are plotted and show that the peak resonant frequency of the probe tip in mechanical contact with the cement sample varies from about 150 Hz at the start to about 220 Hz at the end of the test. During the first approximately 400 seconds of the test duration, the cement goes from a runny liquid state to a thick viscous dough state, after which the dough is to firm to use and lacks stickiness. During this curing period of 400 seconds, the temperature of the cement only changes about 1° C. The small change in temperature, clearly, is not a reliable indicator of the state of cure of the cement, whereas the peak resonant frequency changes by about 16 Hz and is easily discernable. If we plot the peak resonance frequency as a function of time as shown by the circle markers in FIGS. 4, 5, and 6, we can see that during the first 400 seconds, the change in frequency versus time is linear in behavior and a linear curve fit for the first 400 seconds gives the slope indicated: 0.037 Hz/sec for FIG. 4, 0.0433 Hz/sec for FIG. 5, and 0.0458 Hz/sec for FIG. 6. FIGS. 4, 5, and 6 also shows that the first 400 seconds, the bulk cement temperature as monitored using an embedded negative temperature coefficient (NTC) 10 k ohm thermistor shows that the temperature only rises about 1° C. Furthermore, we can see that the resonance frequency continues to vary linearly beyond the usable cement cure time of 400 seconds until the frequency peak shifts at an accelerated rate until it changes no more (not shown) when the sample has fully cured and is hard. The lack of change of resonance frequency can also be used as a positive indicator of the cement reaching the final cure state.

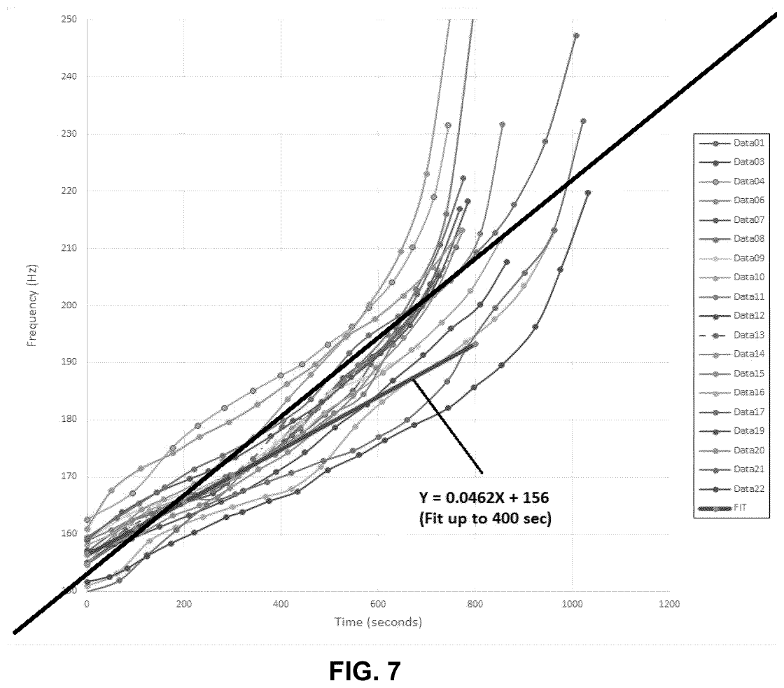

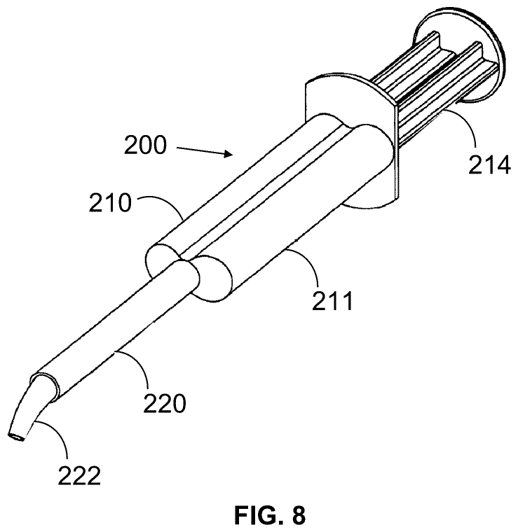

FIG. 7 shows the data from 19 separate tests all plotted on the same graph and the average frequency shift versus time slope has been calculated and shown as having a value of 0.0462 Hz/sec+/−0.00624 Hz/sec or a 13.5% standard deviation. This shows that for all mixtures and tests, the progress of the PMMA cure can be tracked using this slope and when a change in frequency of about 16 Hz has been detected, the sample has reached the point of non-usability and must be discarded. So detecting a 4 Hz shift for example, would indicate that the cement has reached 25% of its usable life while it is awaiting inside the applicator syringe. Similarly, detecting a 12 Hz frequency shift would indicate that the PMMA cement has progressed about 75% through its usable life as it awaits use in the applicator syringe. Now, for the first time, a simple and quantitative means of monitoring the in-situ progress of the PMMA cement cure inside the applicator syringe or mixing chamber is possible. FIG. 1 illustrates a prototype of the invention with a syringe handle and piston that contains the voice coil actuator and piezoelectric transducer sensors for the real-time and in-situ monitoring of the PMMA cure progress inside a disposable applicator syringe. FIG. 8 shows an applicator 200 in which the components are provided in separate compartments 210, but which can be ruptured by a plunger 214 and feed through a spiral mixing container 220 to agitate and combine the components. In this mixing compartment 220, the state of the mixture is monitored to determine the appropriate state for use, and the applicator includes an applicator nozzle 222 through which the mixture is feed for use. This applicator provides a closed system such as to confine noxious fumes and to reduce the mess and trouble that can be associated with the individual components and with the adhesive mixture. The applicator system can be provided in a sterilized or sterilizable state.

### Theory and Analysis

PMMA undergoes an exothermic reaction during cure. During the phase transition of PMMA from liquid to solid (curing) the exothermic reaction and this generates heat that causes the bulk temperature of the cement to increase. This temperature increase however, is not as easily monitored as the shift in resonance frequency of the mechanical structure formed by the transducer voice coil/piezoelectric transducer and probe tip which is in contact with the cement. As the cement cures, its viscosity or stiffness under mechanical strain increases. This mechanical property modifies the resonance frequency of the vibrating probe tip in such a way that is easily measured and distinguished using a FFT to determine the resonance frequency peak.

One theory as to this shift follows: In its simplest form, the resonance structure of the probe tip can be thought of as a simple cantilever beam is given by the equation:

$$f = \frac{Kn}{2 π} \sqrt{\frac{EIg}{{wl}^{4}}}$$

Where Kn is the mode of vibration, E is Young's modulus, l is the area moment of inertia, g is the gravitational acceleration, w is the beam width, and l is the beam length. Having the tip of the beam in contact with the viscous cement will essentially be affecting the beam's apparent Youngs modulus and this in turn, modifies the resonant frequency. We can see that a stiffer cement will increase the Youngs modulus and cause the resonant frequency to increase with increasing viscosity or stiffness of the dough state. We can also see why it is so sensitive because the viscosity is applied to the tip of the cantilever beam so that has an effect that is proportional to the 4^{th} power of the length of the beam. There are other resonance structures that may be even more sensitive or can be designed to be more advantageous (such as discs with torsional springs or other 3D mechanical resonant structures such as a tuning fork). These other embodiments are just variants of the main technique taught here: that the change in the viscosity of the PMMA cement can be detected and monitored by the change in the resonant frequency of a structure in mechanical contact with the cement sample. Regardless of this theoretical underpinning, it has been shown empirically, and statistical significance that the present invention works.

Although the present invention has been described based upon the above embodiments and the data produced by measurement of the performance of the resulting invention that has been reduced to practice, it is apparent to those skilled in the art that certain modifications, variations, and alternative constructions would be apparent, while remaining within the spirit and scope of the invention. In order to determine the metes and bounds of the invention, reference should be made to the following claims.

What is claimed is:

1. A system for sensing the state of cure progress of PMMA cement, comprising:
a applicator for use during a surgery having a reservoir that contains a quantity of a cement during cure, a sensor assembly having a sensor probe which is in contact with the quantity of cement and which monitors one or more of the mechanical resonance spectra and frequency peaks of the sensor probe in mechanical contact with the quantity of cement and a signal processing system which is in communication with the sensor probe to determine and communicate a state of cure of the cement using one or more of either the mechanical resonance spectra and the frequency peaks of the sensor probe.

2. A system for sensing the cure progress of PMMA cement as set forth in claim 1, wherein the sensor assembly includes a first electromechanical transducer in mechanical series contact with a second electromechanical transducer in mechanical series contact with the sensor probe which has a distal tip in contact with the quantity of cement.

3. A system for sensing the cure progress of PMMA cement as set forth in claim 2, wherein the first electromechanical transducer acts as a mechanical excitation or transmitter of mechanical vibrations.

4. A system for sensing the cure progress of PMMA cement as set forth in claim 3, wherein the second electromechanical transducer acts as a mechanical excitation receiver of vibrations.

5. A system for sensing the cure progress of PMMA cement as set forth in claim 4, wherein the first and second electromechanical transducers in mechanical contact and in series with the distal tip of the sensor probe form a sensor probe assembly with a self-resonant frequency spectrum and natural frequency peaks.

6. A system for sensing the cure progress of PMMA cement as set forth in claim 1, wherein the hardness of the quantity of cement represents its state of cure and this hardness in mechanical contact with the distal tip of the sensor probe, modifies the natural resonance of spectra of the sensor probe assembly.

7. A system for sensing the cure progress of PMMA cement as set forth in claim 6, wherein the hardness modifies the resonance frequency of the sensor probe assembly by shifting the resonance frequency.

8. A system for sensing the cure progress of PMMA cement as set forth in claim 7, wherein the sensor probe is in contact with the quantity of cement during cure.

9. A system for sensing the cure progress of PMMA cement as set forth in claim 1, wherein the sensor assembly comprises electromechanical transducers which are electromagnetically actuated voicecoil motors.

10. A system for sensing the cure progress of PMMA cement as set forth in claim 1, wherein the sensor assembly comprises electromechanical transducers which are electrically actuated piezoelectric crystals.

11. A system for sensing the cure progress of PMMA cement as set forth in claim 1, wherein the sensor assembly comprises a plurality of electromechanical transducers in series which include an electromagnetically actuated voicecoil motor and an electrically actuated piezoelectric crystal transducer.

12. A system for sensing the cure progress of PMMA cement as set forth in claim 11, wherein the sensor assembly includes a dual voicecoil transducer or a dual PZT transducer.

13. A system for sensing the cure progress of PMMA cement as set forth in claim 1, wherein the sensor assembly uses a sweep signal can be in the audio frequencies of 20 Hz to 20 KHz.

14. A system for sensing the cure progress of PMMA cement as set forth in claim 2, wherein the applicator further includes a piston and the piston includes the distal tip of the sensor probe.

15. A surgical method, comprising:
the steps of mixing the components of a bone cement and containing a quantity of the mixed cement within a surgical applicator during cure and monitoring the cure of the bone cement using a sensor assembly having a sensor probe which is in contact with the quantity of cement which monitors one or more of the mechanical resonance spectra and frequency peaks of the sensor probe in mechanical contact with the quantity of cement and a signal processing system which is in communication with the sensor probe to determine and communicate a state of cure of the cement based either the mechanical resonance spectra or the frequency peaks of the sensor probe.
